(19) United States
(12) Patent Application Publication
(10) Pub. No.: US 2016/0022253 A1
(43) Pub. Date: Jan. 28, 2016

(54) ENDOSCOPIC CLOSURE DEVICE USING SEALANTS

(71) Applicant: Boston Scientific Scimed, Inc., Maple Grove, MN (US)

(72) Inventors: Priya Khanchandani, Marlborough, MA (US); Paul Smith, Smithfield, RI (US); Kevin Wilcox, Brighton, MA (US); Shawn Kerr, Lancaster, MA (US)

(21) Appl. No.: 14/801,344

(22) Filed: Jul. 16, 2015

Related U.S. Application Data
(60) Provisional application No. 62/028,148, filed on Jul. 23, 2014.

(51) Int. Cl. A61B 17/00; A61B 17/08; A61B 17/12; A61B 17/11

(52) U.S. Cl. CPC A61B 17/0057; A61B 17/00491; A61B 17/00234; A61B 17/1114; A61B 17/083; A61B 17/1214; A61B 2017/00296; A61B 2017/00495; A61B 2017/00818; A61B 2017/00575; A61B 2017/0065; A61B 2017/00623

(57) ABSTRACT

A system for treating a tissue defect includes a delivery catheter extending longitudinally from a proximal end to a distal end and including a channel extending therethrough and a tissue closure device movably received with the channel of the delivery catheter to be delivered to target tissue to be treated in combination with a lumen extending within the channel to pass a sealant to target tissue.

### PRIORITY CLAIM

The present disclosure claims priority to U.S. Provisional Patent Application Ser. No. 62/028,148 filed Jul. 23, 2014; the disclosure of which is incorporated herewith by reference.

### BACKGROUND

Physicians have become increasingly willing to perform more aggressive interventional and therapeutic endoscopic procedures including, for example, removal of larger lesions (i.e., cancerous masses), tunneling under mucosal layers in the gastro-intestional (GI) tract to treat tissues below the mucosa, full thickness removal of tissue, inserting devices through the GI tract and then penetrating the GI organ to treat tissue outside the GI tract, and endoscopic treatment/repair of post-surgical issues (e.g., post-surgical leaks, breakdown of surgical staple lines, anastomotic leaks). These procedures may increase the risk of perforating the wall of the GI tract, or may require closure of the GI tract wall as part of the procedure. In some cases, inadvertent perforations may not extend through an entire tissue wall. Mucosal defects may not be completely open, having one or more layers (e.g., submucosal, serosa, muscle) still intact. Endoscopic closure reduces cost and may reduce the trauma and inconvenience associated with these procedures. However, current tissue closure devices may be insufficient to close certain perforations and/or defects.

### SUMMARY

The present disclosure comprises system for treating a tissue defect, comprising a delivery catheter extending longitudinally from a proximal end to a distal end and including a channel extending therethrough and a tissue closure device movably received within the channel of the delivery catheter to be delivered to target tissue to be treated in combination with a lumen (e.g., a sealant lumen) extending through the delivery catheter, particularly, within the channel thereof, to pass a sealant to target tissue.

In an embodiment, the tissue closure device may be a coil extending along a helical path about a longitudinal axis thereof.

In an embodiment, the helical path may define the lumen through which the sealant is applied.

In an embodiment, the system may further comprise an inner sleeve extending within the channel longitudinally from a proximal end to a distal end, the lumen extending longitudinally through the inner sleeve.

In an embodiment, the tissue closure device may be a clip movably received within the channel.

In an embodiment, the clip may include a plurality of arms movable between an open configuration, in which distal ends of the arms are separated from one another to receive tissue therebetween, and a closed configuration, in which distal ends of the arms are drawn toward one another to grip tissue received therebetween.

In an embodiment, the inner sleeve may be longitudinally movable within the channel

In an embodiment, the delivery catheter may include an injection port at the proximal end thereof for connecting to a unit housing the sealant.

In an embodiment, the sealant may include one of Fibrin and polyethylene glycol.

The present disclosure is also directed to a device for delivering a sealant to a target tissue area, comprising a catheter extending along a longitudinal axis from a proximal end to a distal end and including a channel extending therethrough, and a distal portion connected to the distal end of the catheter and sized and shaped to deliver a sealant passed through the channel to the distal portion to a defined area of a target tissue.

In an embodiment, the distal portion may flare from a proximal end thereof toward a distal end thereof to deliver the sealant to the defined area, the defined area being larger than a cross-sectional area of a portion of the catheter extending proximally of the distal portion.

In an embodiment, the flared distal portion may be formed of a compliant material such that the distal portion is constrained to a reduced cross-section by an interior surface of a working channel while it is received therein.

In an embodiment, the distal portion may taper from a proximal end thereof to a distal end thereof such that the sealant is delivered to the defined area, the defined area being smaller than a cross-sectional area of the catheter extending proximally of the distal portion.

In an embodiment, the device may further comprise an injection port at the proximal end of the catheter, the injection port configured to be coupled to a unit housing a sealant material.

In an embodiment, the device may further comprise a controller coupled to the injection port for controlling a flow of sealant through the channel.

The present disclosure is also directed to a method for treating a tissue defect including inserting a delivery catheter to a target tissue via a channel of an endoscope, moving a tissue closure device housed within a delivery catheter channel distally relative to the delivery catheter such that the tissue closure device extends distally past a distal end of the delivery catheter to extend over a portion of the target tissue and applying a sealant to the target tissue via the delivery catheter channel to the target tissue via the delivery catheter.

### BRIEF DESCRIPTION OF THE DRAWINGS

FIG. 1 shows a perspective view of a device according to a first exemplary embodiment of the present disclosure, in a first configuration;

FIG. 2 shows an enlarged perspective view of the device of FIG. 1;

FIG. 3 shows an enlarged cross-sectional view of a distal portion of the device of FIG. 1;

FIG. 4 shows a perspective view of a distal portion of a device according to a second exemplary embodiment of the present disclosure;

FIG. 5 shows a side view of a device according to a third exemplary embodiment of the present disclosure;

FIG. 6 shows an enlarged side view of the device of FIG. 5; and

FIG. 7 shows a side view of a distal portion of a device according to a fourth exemplary embodiment of the present disclosure.

### DETAILED DESCRIPTION

The present disclosure may be further understood with reference to the following description and the appended drawings, wherein like elements are referred to with the same reference numerals. The present disclosure related to devices for tissue closure and, in particular, to an endoscopic tissue closure device. Exemplary embodiments of the present disclosure describe an endoscopic tissue closure device comprising a clip or coil along with a sealant applied over portions of tissue being drawn together to close a tissue defect. It should be noted that the terms proximal and distal as used herein, are intended to refer to a direction toward (proximal) and away (distal) from a user of the device.

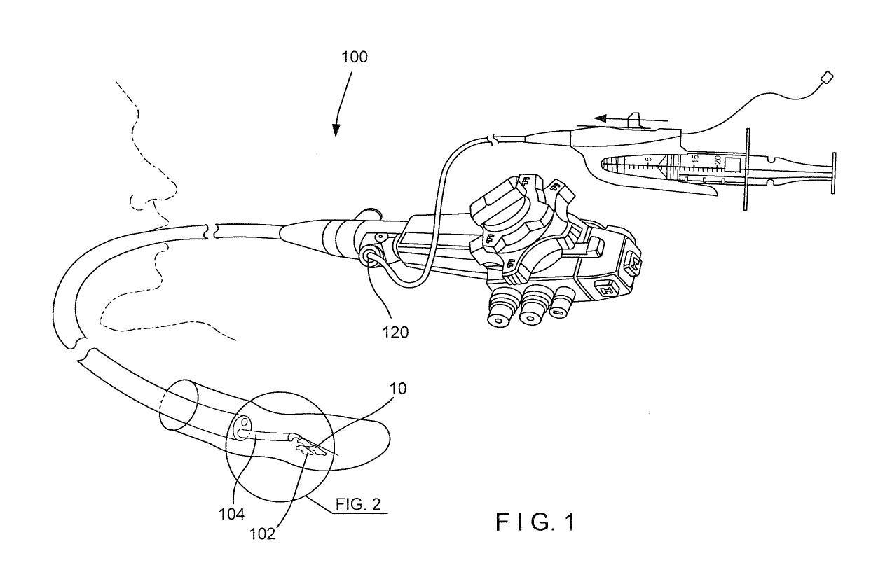

As shown in FIGS. 1 - 3, a device 100 according to an exemplary embodiment of the present disclosure comprises a coil 102 passed through a delivery catheter 104 in combination with a sealant material 106 (e.g., cyanoacrylate) to close a tissue defect 10. The coil 102 extends along a substantially helical path from a proximal end 108 to a distal end 110 so that a hollow core 112 is defined thereby. In one embodiment, the hollow core may be substantially cylindrical. In other embodiments, the hollow core 112 may have any of a variety of shapes so long as the coil 102 may be passed through a channel of the delivery catheter 104. In one embodiment of the present disclosure, the coil 102 may be passed distally through a channel 114 of the delivery catheter 104 such that the distal end 110 of the coil 102 may be positioned over a first portion of tissue along a first side 12 of the tissue defect 10 while the proximal end 108 is positioned over a second portion of tissue along a second side 14 of the tissue defect 10 substantially opposing the first side. The coil 102 may act as a scaffold bridging the first and second sides 12, 14 of the tissue defect 10. In another embodiment, the adjacent turns of the coil 102 at the proximal and distal ends 108, 110 grip a portion of tissue along the first and second sides 12, 14, approximating the first and second sides 12, 14. In another embodiment, a plurality of coils 102 may be deployed in the tissue defect 10 in a random fashion such that the coils 102 fill the defect 10 to provide a webbed matrix bridging or scaffolding the wound. This embodiment may be particularly suited for treating mucosal defects in which some layers have not been perforated and are intact. The coil 102 in combination with the sealant material 106 prevents delayed bleeding and the potential for perforation.

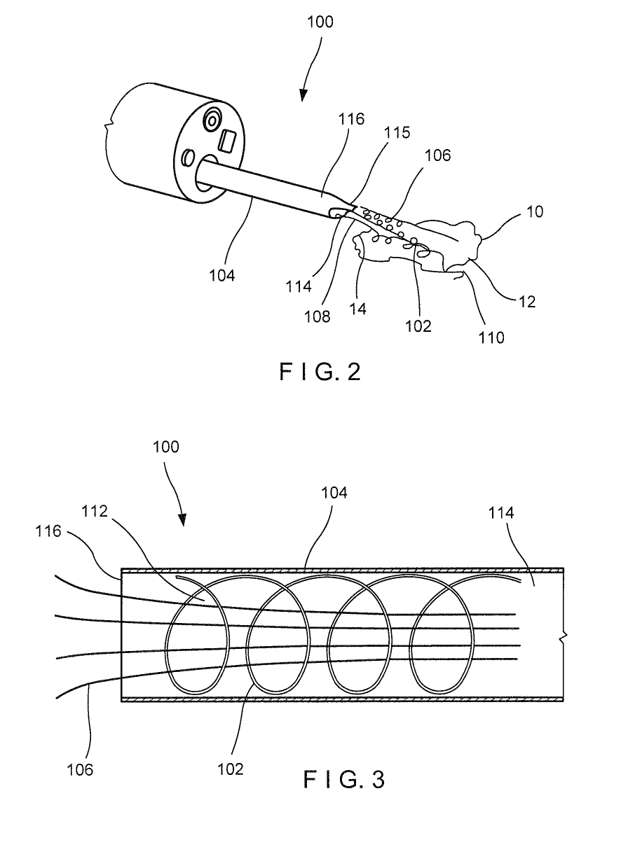

In one embodiment, as shown in FIG. 2, the sealant material 106 is provided via a separate lumen 115 (e.g., a sealant lumen) of the delivery catheter 104 to be applied simultaneously with the coil 102 and/or separately before and/or after deployment of the one or more coils 102 in the defect 102. In another embodiment, as shown in FIG. 3, the sealant material 106 is passed through the channel 114 of the catheter 104 so that the sealant material 106 is applied over the tissue defect 10 as the coil 102 is deployed from a distal end of the delivery catheter 104 to engage the first and second portions of tissue. The sealant material 106 therefore extends over the tissue defect 10 from the first portion of tissue 12 to the second portion of tissue 14 along the coil 102 to close the tissue defect 10. The coil 102 may be deployed from the delivery catheter 104 via, for example, a control rod which pushes the coil 102 distally out of the channel 114. As described above, the device 100 may include a plurality of coils 102 to bridge opposing sides of the tissue defect 10 along a length thereof. The combination coil 102 and sealant material 106 will provide physicians the ability to close varying size defects such as fistula, pockets and bleeds and provided an easy to use technique to directly apply the sealant to the target site to achieve closure.

The coil 102 may be formed, for example, of an elastic metal to be applied over the tissue defect 10 as described above. A diameter of the coil 102 may, for example, be constrained by a surface of the channel 114 when received therein and permitted to expand under a natural bias of the coil 102 as the coil 102 is moved distally beyond a distal end 116 of the catheter 104. A length of the coil 102 may be selected to substantially correspond to a width of a tissue defect 10 to be treated (e.g., a distance between the first and second portions on opposing sides 12, 14 of the tissue defect 10). The length of the coil 102 may vary, however, as proximal and distal ends 108, 110 of the coil 102 are not required to engage the opposing sides 12, 14. As described above, the coil(s) 102 may be used to fill the defect 10 to form a webbed matrix. Furthermore, a diameter of the coil 102 may be substantially constant along a length thereof or may vary in any manner as desired.

The delivery catheter 104 includes an elongate body extending longitudinally from a proximal end (not shown) to the distal end 116 and includes the channel 114 extending therethrough. The channel 114 is sized and shaped to receive the coil 102 therein such that the hollow core 112 defined by the helical shape of the coil 102 is substantially aligned with a longitudinal axis of the channel 114. The delivery catheter 104 may also include a lumen 115 (e.g., a sealant lumen) for separately delivering the sealant material 106 to the tissue defect 10. The delivery catheter 104 also includes an injection port 120 at the proximal end 118 through which the sealant material 106 may be passed into the lumen 115. The injection port 120 is configured, for example, to be connected to a unit which houses the sealant material 106 therein. The injection port 120 may, for example, include barbs or other connecting features for connecting to the unit housing the sealant material 106. In another embodiment, the sealant material 106 passed into the channel 114 via the injection port 120 so that the sealant material 106 may be received within the hollow core 112 of the coil 102 to be applied at the same time that the coil 102 is being applied across the tissue defect 10.

The sealant material 106 may include materials such as, for example, Fibrin or Polyethylene Glycol (PEG), which have been shown to have good clinical efficacy in the treatment of tissue defects. Fibrin, in particular, is biocompatible and biodegradable and is advantageous over synthetic polymers and collagen gels in terms of inflammation, immune responses, toxicity and cell adhesion.

According to an exemplary surgical technique using the device 100, the delivery catheter 104, including the coil 102 housed within the channel 114 thereof, is passed through a working channel of an endoscope to a target area within a living body, for example, along a body lumen accessed via a naturally occurring body orifice. The distal end 116 of the catheter 104 is positioned proximate the tissue defect 10 to be treated. Once the delivery catheter 104 has been positioned, as desired, the coil 102 is pushed distally past the distal end 116 of the catheter 104 so that the distal end 110 of the coil 102 is positioned over the first portion of tissue 12 on the first side of the tissue defect 10. As the coil 102 is being delivered out of the catheter 104, the distal end 116 of the catheter 104 is moved from the first portion of tissue 12 toward the second portion of tissue 14 along the second side of the tissue defect 10 so that the proximal end 108 of the coil 102 is positioned over the second portion of tissue 14 and the sealant material 106 is, for example, simultaneously applied along the coil 102. Thus, the deployed coil 102 forms a bridge over the tissue defect 10 from the first side to the second side of the tissue defect 10 with the sealant material 106 extending therealong. More than one coil 102 and the sealant material 106 combination may be deployed across the tissue defect 10 from the first side 12 to the second side 14 to seal the tissue defect 10. For example, multiple coils 102 may be positioned in the catheter sequentially with a proximal end of one coil 102 abutting a distal end of an adjacent coil 102. Thus, as a proximal-most one of the coils 102 is pushed distally through the catheter 104, the next more distal coil 102 is moved distally until the distal-most coil 102 is moved out of the catheter 104 as desired. After this distal most coil 102 has been ejected from the catheter 104, the adjacent coil 102 which is now the distal-most coil 102 remaining in the catheter 104 may be deployed at a new site by simply repositioning the catheter 104 and repeating this process.

In another embodiment, as described above the coil 102 may be delivered into the tissue defect 10 in a random fashion so that multiple coils 102 together may fill the tissue defect 10, forming a webbed matrix of coils 102. The sealant material 106 may be delivered to the tissue defect 10 simultaneously with or separately after, for example, delivery of a desired number of coils 102 into the tissue defect 102. The coils 102 act as a scaffold, preventing embolization of the sealant material 106.

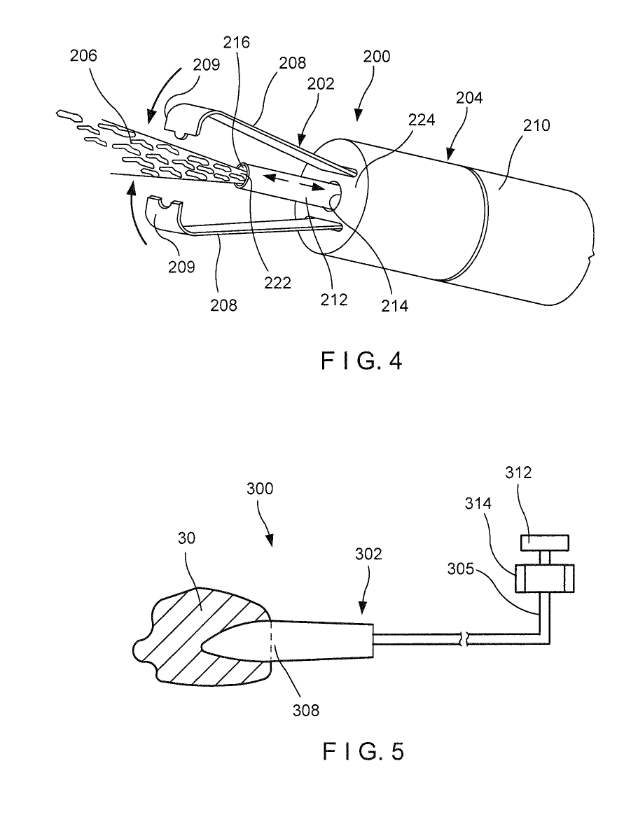

As shown in FIG. 4, a device 200 according to a second exemplary embodiment of the present disclosure is substantially similar to the device 100 described above except as noted. The device 200 comprises a clip 202 rather than a coil 102 as described above in regard to the device 100. The device 200, applies the clip 202 over a tissue defect in combination with a sealant material 206 via a delivery catheter 204. The clip 202 includes a plurality of arms 208 movable between an open configuration, in which distal ends 209 of the arms 208 are separated from one another to receive tissue therebetween, and a closed configuration, in which the distal ends 209 of the arms 208 are moved toward one another to grip the tissue received therebetween. Although the clip 202 is shown and described as including two arms 208, it will be understood by those of skill in the art that the clip 202 may include any number of arms 208 so long as the arms 208 are able to move between the open and closed configurations to grip tissue.

The delivery catheter 204 may be comprised of an outer sleeve 210 and an inner sleeve 212 to form a first lumen 214 and a second lumen 216. In particular, the inner sleeve 212 according to this embodiment extends within the outer sleeve 210 such that the first lumen 214 is defined by an annular space between an interior surface of the outer sleeve 210 and an exterior surface of the inner sleeve 212. The second lumen 216 is defined by an interior surface of the inner sleeve 212. Arms 208 of the clip 202 are movably housed within the first lumen 214 while the sealant material 206 is passed through the second lumen 216 so that the sealant material 206 and the clip 202 may be simultaneously applied over a tissue defect. The sealant material 206, however, is not required to be simultaneously applied to the tissue defect. The sealant material 206 may alternatively or additionally be applied before and/or after application of the clip 202.

Similarly to the delivery catheter 104 of the device 100, the delivery catheter 204 includes an injection port (not shown) at a proximal end thereof. The injection port may be connected to the delivery catheter 204 so that the sealant material 206 may be received therethrough and into the second channel 216 so that the sealant material 206 may be applied over the tissue defect during clipping of the tissue defect. Lengths of the inner and outer sleeves 212, 210, however, may vary. For example, a distal end 222 of the inner sleeve 212 may extend distally of a distal end 224 of the outer sleeve 210. In another embodiment, the inner sleeve 212 may be movable relative to the outer sleeve 210 so that the inner sleeve 212 may be moved distally relative to the outer sleeve 210 to spray the tissue defect, then retracted into the outer sleeve 210 after completion of the spraying.

Proximal ends of the clip arms 208 in this embodiment are connected to one another and include an opening or space extending therethrough for accommodating the second lumen 216, when the clip 202 is housed within the first lumen 214. In one embodiment, the clip arms 208 are biased toward the open configuration so that, when the clip 202 is received within the first lumen 214, the arms are constrained toward the closed configuration via the interior surface of the first lumen 214. Once the delivery catheter 204 is positioned proximate the target tissue, however, the clip 202 is moved distally out of the first lumen 214 freeing the arms 208 to revert to the open configuration to receive the target tissue therebetween. The arms 208 may then be moved to the closed configuration by sliding a locking element (e.g., a locking ring) over the arms 208 to move the arms 208 to the closed configuration and lock the arms 208 in the closed configuration. In another embodiment, the clip 202 may be biased toward the closed configuration so that the arms 208 are permitted to revert to the closed configuration over the target tissue when completely deployed from the first lumen 214. In particular, the delivery catheter 204 may include features for holding the arms 208 in the open configuration when distal ends 209 of the arms 208 are moved distally past the distal end 224 of the first lumen 214. Once the target tissue is received between the arms 208, the clip 202 may be moved further distally relative to the first lumen 214, disengaging the arms 208 from the holding feature and permitting the arms 208 to revert to the biased closed configuration over the target tissue.

According to a surgical technique using the device 200, the delivery catheter 204 may be inserted to a target area within a patient body via a working channel of an endoscope. The inner sleeve 212 may be moved distally relative to the outer sleeve 210 such that the distal end 222 of the inner sleeve 212 extends distally past the distal end 224 of the outer sleeve 210 to spray the sealant material 206 into the tissue defect. Upon spraying the sealant material 206 into the tissue defect. The inner sleeve 212 may be retracted into the outer sleeve 210 or entirely withdrawn therefrom. Distal end 214 of the delivery catheter 204 is positioned proximate the tissue defect to be closed, the clip 202 is moved distally relative to first lumen 214 via, for example, a pusher rod (not shown) extending within the first lumen, so that the clip arms 208 are moved to the open configuration to receive the target tissue therebetween. Each of the arms 208 may be positioned over portions of tissue on opposing sides of the tissue defect. Once the target tissue has been positioned between the arms 208, as desired, the clip 202 may be moved to the closed configuration to grip the tissue therebetween. The application of the clip 202 and the sealant material 206 ensures an effective closure of the tissue defect. Although the exemplary surgical technique describes applying the sealant material 206 prior to the application of the clip 202, the sealant material 206 may also be applied after the application of the clip 202 or simultaneously therewith.

As shown in FIGS. 5 - 6, a device 300 according to a third exemplary embodiment of the present disclosure comprises a delivery catheter 302 for delivering a sealant material 306 to a tissue defect 30. The delivery catheter 302 extends longitudinally from a proximal end 305 to a distal end 308 and includes a channel 310 extending therethrough. The delivery catheter 302 is sized and shaped to be inserted to a target area in a patient body via, for example, a working channel of an endoscope. The proximal end 305 further includes an injection port 312 configured to be connected to a unit which houses the sealant material 306 and a controller 314 for controlling the delivery of the sealant material 306. For example, the controller 314 may control an amount of the sealant material 306 that is permitted to pass through the injection port 312 into the channel 310. The sealant material 306 may be substantially similar to the sealant material 106, 206 described above in regard to the devices 100, 200.

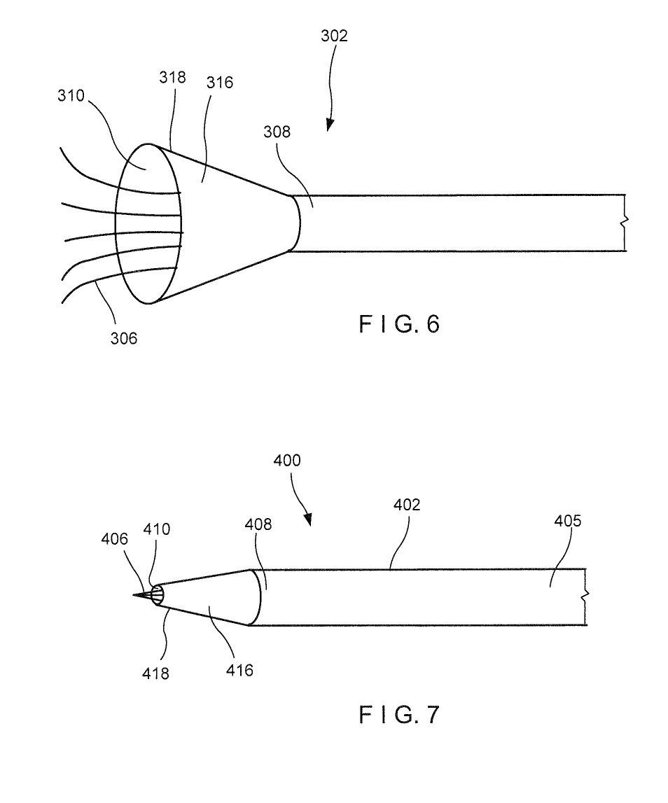

As shown in FIG. 6, the catheter 302 further includes a flared portion 316 at the distal end 308, the flared portion 316 flaring outward toward a distal end 318 thereof facilitates spraying of the sealant material 306 over a larger area. The flared portion 316 is formed of or treated with a lubricious material so that the sealant material 306 may be sprayed therethrough in a confined location patch and is thus particularly suited to treat larger tissue defects. At least the flared portion 316 is formed a compliant material so that the flared portion 316 may be constrained by an interior surface of the working channel when inserted therethrough and permitted to revert to a biased flared configuration when the flared portion 316 is moved distally past a distal end of the working channel. It will be understood by those of skill in the art that the device 300 may be utilized with other endoscopic devices such as clipping devices and/or coils to apply a sealant to a tissue defect as the tissue defect is also being treated using other tissue closure devices. It will also be understood by those of skill in the art that either of the delivery catheter 104, 204 described above in regard to the devices 100, 200 may include the flared portion 316 described in regard to the device 300. For example, the distal end 116 of the delivery catheter 104 may include a flared portion to spray the sealant material 106 in a confined location patch.

As shown in FIG. 7, a device 400 according to a fourth exemplary embodiment of the present disclosure may be substantially similar to the device 300 described above, comprising a delivery catheter 402 for delivering a sealant material 406 to a tissue defect to be closed. The delivery catheter 402, however, provides more controlled delivery to the target tissue defect. Similarly to the delivery catheter 302, the catheter 402 extends longitudinally from a proximal end 405 to a distal end 408 and includes a channel 410 extending therethrough, through which the sealant material 406 is passed. Rather than a flared portion, however, the catheter 402 includes a tapered distal portion 416 which delivers the sealant material 406 at pin-point location. In particular, the distal portion 416 is funnel-shaped, tapering towards a distal end 418 thereof. Similarly to the device 300, the device 400 may be utilized with other endoscopic tissue closure devices. It will be understood by those of skill in the art that either of the deliver devices 104, 204 described above in regard to the devices 100, 200 may include the tapered distal portion 416 described in regard to the device 400. For example, the distal end 222 of the inner sleeve 212 of the delivery catheter 204 may include the tapered portion to deliver the sealant material 206 with pin-point precision.

It will be apparent to those skilled in the art that various modifications may be made in the present disclosure, without departing from the scope of the disclosure. Thus, it is intended that the present disclosure cover modifications and variations of this disclosure provided that they come within the scope of the appended claims and their equivalents.

1-15. (canceled)

16. A system for treating a tissue defect, comprising:
a delivery catheter extending longitudinally from a proximal end to a distal end and including a channel extending therethrough;
a tissue closure device movably received with the channel of the delivery catheter to be delivered to target tissue to be treated; and
a lumen extending through the delivery catheter to pass a sealant to target tissue.

17. The system of claim 16, wherein the tissue closure device is a coil extending along a helical path about a longitudinal axis thereof.

18. The system of claim 17, wherein the helical path defines the lumen through which the sealant is applied.

19. The system of claim 16, further comprising an inner sleeve extending within the channel longitudinally from a proximal end to a distal end, the lumen extending longitudinally through the inner sleeve.

20. The system of claim 19, wherein the tissue closure device is a clip movably received within the channel.

21. The system of claim 20, wherein the clip includes a plurality of arms movable between an open configuration, in which distal ends of the arms are separated from one another to receive tissue therebetween, and a closed configuration, in which distal ends of the arms are drawn toward one another to grip tissue received therebetween.

22. The system of claim 19, wherein the inner sleeve is longitudinally movable within the channel.

23. The system of claim 16, wherein the delivery catheter includes an injection port at the proximal end thereof for connecting to a unit housing the sealant.

24. The system of claim 16, wherein the sealant includes one of Fibrin and polyethylene glycol.

25. A device for delivering a sealant to a target tissue area, comprising:
a catheter extending along a longitudinal axis from a proximal end to a distal end and including a channel extending therethrough; and
a distal portion connected to the distal end of the catheter and sized and shaped to deliver a sealant passed through the channel to the distal portion to a defined area of a target tissue.

26. The device of claim 25, wherein the distal portion flares from a proximal end thereof toward a distal end thereof to deliver the sealant to the defined area, the defined area being larger than a cross-sectional area of a portion of the catheter extending proximally of the distal portion.

27. The device of claim 26, wherein the flared distal portion is formed of a compliant material such that the distal portion is constrained to a reduced cross-section by an interior surface of a working channel while it is received therein.

28. The device of claim 25, wherein the distal portion tapers from a proximal end thereof to a distal end thereof such that the sealant is delivered to the defined area, the defined area being smaller than a cross-sectional area of the catheter extending proximally of the distal portion.

29. The device of claim 25, further comprising an injection port at the proximal end of the catheter, the injection port configured to be coupled to a unit housing a sealant material.

30. The device of claim 25, further comprising a controller coupled to the injection port for controlling a flow of sealant through the channel.

31. A method for treating a tissue defect, comprising:
inserting a delivery catheter to a target tissue via a channel of an endoscope;
moving a tissue closure device housed within a delivery catheter channel distally relative to the delivery catheter such that the tissue closure device extends distally past a distal end of the delivery catheter to extend over a portion of the target tissue; and
applying a sealant to the target tissue via the delivery catheter channel to the target tissue via the delivery catheter.

32. The method of claim 31, wherein the tissue closure device is a coil extending along a helical path about a longitudinal axis thereof.

33. The method of claim 32, wherein moving the tissue closure device includes positioning a distal end of the coil over a first portion of tissue along a first side of a tissue defect and a proximal end of the coil over a second portion of tissue along a second side of the tissue defect such that the coil forms a bridge across the tissue defect.

34. The method of claim 31, wherein the delivery catheter includes an outer sleeve extending longitudinally from a proximal end to a distal end and an inner sleeve extending longitudinally from a proximal end to a distal end, the inner sleeve housed within the outer sleeve to form a first lumen defined by an interior surface of the outer sleeve and an exterior surface of the inner sleeve and a second lumen defined by an interior surface of the inner sleeve.

35. The method of claim 34, wherein the tissue closure device is a clip including a plurality of arms movable between an open configuration, in which distal ends thereof are separated from one another, and a closed configuration, in which the distal ends thereof are drawn toward one another, and the sealant is received within the second lumen so that the sealant is applied to the target tissue between the arms of the clip.
